(19) United States
(12) Patent Application Publication
(10) Pub. No.: US 2007/0053864 A1
(43) Pub. Date: Mar. 8, 2007

(54) Poly(potassium and sodiumstyrene sulfonate) its manufacture and its uses

(76) Inventors: Caroline Bacon-Kurtz, Sudbury, MA (US); David Davidson, Lincoln, MA (US)

Correspondence Address: Attention: Christopher M. Beck;Legal Department, 153 Second Avenue, Waltham, MA, 02451, US

(21) Appl. No.: 11/488,896

(22) Filed: Jul. 18, 2006

Related U.S. Application Data
(63) Continuation of application No. 11/039,351, filed on Jan. 20, 2005, now abandoned.
Application No. 11/039,351 is a continuation of application No. PCT/US03/22514, filed on Jul. 18, 2003.
(60) Provisional application No. 60/397,868, filed on Jul. 22, 2002.

(51) Int. Cl. A61K 38/16; C08J 5/20; A61K 31/74

(52) U.S. Cl. 424/78.1; 514/8; 514/396; 521/25

(57) ABSTRACT

Antibiotic-associated diarrhea, such as that caused by Clostridium difficile, represents a serious medical complication that can result from administering a broad-spectrum antibiotic to a subject. Such diarrhea leads to significant potassium loss from the subject. The present invention discloses a polymeric therapeutic agent that treats antibiotic-associated diarrhea and is physiologically potassium neutral. This polymer contains polystyrene sodium sulfonate and polystyrene potassium sulfonate repeat units.

### RELATED APPLICATION

This application is a Continuation of U.S. application Ser. No. 11/039,351 filed on Jan. 20, 2005, which is a continuation of International Application No. PCT/US2003/022514, which designated the United States and was filed on Jul. 18, 2003, published in English, which claims the benefit of U.S. Provisional Application No. 60/397,868 filed on Jul. 22, 2002.

### BACKGROUND OF THE INVENTION

Many pathogens produce toxins which are detrimental, and in some cases, lethal, to the host organism. Toxins produced by pathogens can be classified into two general categories, exotoxins and endotoxins. Exotoxins are generally proteins or polypeptides secreted by a pathogen. Endotoxins are lipopolysaccharides or lipoproteins found in the outer layer of the cell walls of gram-negative bacteria.

Each type of toxin is associated with a number of symptoms. Endotoxins may cause fever, diarrhea, vomiting, and decreases in lymphocyte, leukocyte, and platelet counts. Exotoxins may cause hemolysis, septic shock, destruction of leukocytes, vomiting, paralysis, and diarrhea. A class of exotoxins, the enterotoxins, act on the small intestine and cause massive secretion of fluid into the intestinal lumen, leading to diarrhea. Enterotoxins are produced by bacteria such as Clostridium difficile, Clostridium perfringens, Clostridium sordelli, Staphylococcus aureus, Bacillus cereus, Vibrio cholerae, Escherichia coli, and Salmonella enteritidis.

Clostridium difficile has become one of the most common nosocomially-acquired organisms in hospitals and long term care institutions. The organism typically infects patients whose normal intestinal flora has been disturbed by the administration of a broad-spectrum antibiotic. The diarrhea and inflammatory colitis associated with infection represent a serious medical and surgical complication leading to increased morbidity and mortality, and prolonging hospital stays by an average of nearly three weeks. This is especially true for the elderly and for patients with serious underlying diseases who are the most likely to develop the infection.

Currently, many treatments for antibiotic-associated diarrhea (AAD) such as C. difficile associated diarrhea are inadequate. Such treatments include discontinuing the antibiotic that caused AAD to manifest and allow the normal colonic flora to recover as rapidly as possible. In most cases, however, that is not sufficient and yet another antibiotic, such as metronidazole or vancomycin, is used to kill the bacteria. Both of these antibiotics have significant drawbacks, such as a high rate of relapse of AAD and potential selection of multi-drug resistant enterococci and staphylococci.

More promising therapies affect the intestinal damage and inflammation caused by enterotoxins, such as C. difficile Toxins A and B. The toxins produced by C. difficile damage the mucosa and are the etiologic agents responsible for the inflammatory colitis. The therapies involve the use of a negatively-charged polymer to inhibit the enterotoxins produced by bacteria, as described in U.S. Pat. Nos. 6,270,755, 6,290,946, 6,419,914, 6,517,826 and 6,517,827, the entire contents of which are incorporated herein by reference.

Patients experiencing diarrhea are susceptible to significant losses of electrolytes, leading to further morbidity. A therapeutic agent such as an anionic polymer, which does not have the potential to further deplete potassium and other electrolytes, is desirable in this patient population. Therefore, it is advantageous to develop a negatively-charged polymer which is physiologically potassium and sodium neutral and/or to develop a negatively-charged polymer with a potassium content that is pre-selected to result in a desirable and/or advantageous physiologically effect when administered to a subject. Such a therapeutic polymer would prevent further loss of potassium and sodium due to administration of the polymer or have other desirable effects.

### SUMMARY OF THE INVENTION

It has now been found that a polystyrene sulfonate random copolymer comprised of sodium styrene sulfonate and potassium styrene sulfonate repeat units is physiologically potassium and sodium neutral when administered to a subject. It has been additionally found that the polystyrene sulfonate random copolymer inhibits bacterial toxins, such as enterotoxins, thereby treating antibiotic-associated diarrhea (hereinafter “AAD”).

In one embodiment, the present invention is a polystyrene sulfonate copolymer, preferably a random copolymer, or a pharmaceutical composition comprised of a polystyrene sulfonate copolymer, where the copolymer is comprised of repeat units represented by Structural Formula (I): 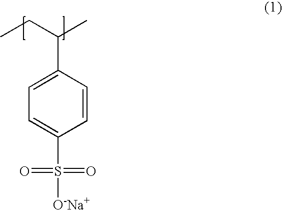
and repeat units represented by Structural Formula (II):

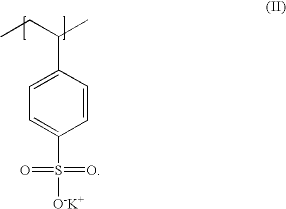

In another embodiment, the present invention is a mixture of sodium polystyrene sulfonate and potassium polystyrene sulfonate or a pharmaceutical composition comprised of a mixture of sodium polystyrene sulfonate and potassium polystyrene sulfonate. The mixture can be a powder, slurry, suspension, or solution of potassium polystyrene sulfonate and sodium polystyrene sulfonate.

In another embodiment, the present invention is a method of treating AAD, where an effective amount of the copolymer comprised of repeat units represented by Structural Formula (I) and Structural Formula (II) or an effective amount of the mixture sufficient to treat the AAD is administered to a mammal. In the present invention, “treating” AAD refers to inhibiting the onset of AAD in susceptible mammals, prophylactically treating those mammals susceptible to AAD, treating ongoing AAD, and inhibiting the relapse of AAD. A susceptible mammal is a mammal at risk of developing AAD or having a relapse of AAD for any reason, including use of broad spectrum antibiotics that may disrupt the normal flora of the gastrointestinal tract, thereby leading to AAD.

In another embodiment, the present invention is a method of preparing the polystyrene sulfonate copolymer. The polystyrene sulfonate copolymer can be prepared by any one of the following steps: copolymerizing the sodium salt of styrene sulfonate and the potassium salt of styrene sulfonate (preferably randomly copolymerizing the salts, alternatively block copolymerizing the salts or alternately copolymerizing the salts), exchanging a proportion of the sodium ions of polystyrene sodium sulfonate for potassium ions, exchanging a proportion of the potassium ions of polystyrene potassium sulfonate for sodium ions, or sulfonating polystyrene and reacting the resultant polystyrene sulfonic acid with a mixture of basic sodium and potassium salts.

In another embodiment, the mixture of sodium polystyrene sulfonate and potassium polystyrene sulfonate can be prepared by physically mixing together sodium polystyrene sulfonate and potassium polystyrene sulfonate. Acceptable forms of sodium polystyrene sulfonate and potassium polystyrene sulfonate for mixing together include dry forms (e.g., powders), slurries, and solutions.

The present invention has many advantages. The polystyrene sulfonate copolymer and the mixture are typically physiologically potassium and sodium neutral, such that administering the copolymer or the mixture to a mammal results in an insignificant change to potassium and/or sodium levels in the mammal. Also, the compositions used in the methods of the invention are easily prepared using standard techniques of polymer synthesis. The disclosed copolymers and mixtures generally do not interfere with the broad spectrum antibiotics utilized to treat other infections of the body and thus can be used in conjunction with broad spectrum antibiotics. Additionally, the compositions and methods of the present invention can be used as monotherapy to inhibit or prevent the onset of disease, to treat disease after onset, or to inhibit or prevent relapse. Monotherapy in accordance with the invention is particularly advantageous when patients cannot tolerate antibiotic regimens, or when further antibiotic therapy is undesirable (i.e., a patient is not responding to antibiotic therapy). A patient who cannot tolerate antibiotic regimens is a patient for whom an antibiotic treatment for antibiotic associated diarrhea is contraindicated.

### DETAILED DESCRIPTION OF THE INVENTION

Polystyrene sulfonate copolymers of the present invention comprise or consist of repeat units represented by Structural Formula (I) and Structural Formula (II). Preferably, about 20% to about 70% of the repeat units are represented by Structural Formula (II) and about 30% to about 80% of the repeat units are represented by Structural Formula (I). Alternatively, about 30% to about 45% of the repeat units are represented by Structural Formula (II) and about 55% to about 70% of the repeat units are represented by Structural Formula (I), about 35% to about 40% of the repeat units are represented by Structural Formula (II) and about 60% to about 65% of the repeat units are represented by Structural Formula (I), or about 37% of the repeat units are represented by Structural Formula (II) and about 63% of the repeat units are represented by Structural Formula (I). In another alternative, about 53% to about 73% of the repeat units are represented by Structural Formula (I) and about 27% to about 47% of the repeat units are represented by Structural Formula (II), about 58% to about 68% of the repeat units are represented by Structural Formula (I) and about 32% to about 42% of the repeat units are represented by Structural Formula (II), about 60.5% to about 65.5% of the repeat units are represented by Structural Formula (I) and about 29.5% to about 44.5% of the repeat units are represented by Structural Formula (II), or about 62% to about 64% of the repeat units are represented by Structural Formula (I) and about 36% to about 38% of the repeat units are represented by Structural Formula (II).

Similarly, polystyrene sulfonate mixtures of the present invention comprise about 20% to about 70%, about 27% to about 47%, about 30% to about 45%, about 32% to about 42%, about 35% to about 40%, about 36% to about 38%, or about 37% potassium polystyrene sulfonate and about 30% to about 80%, about 53% to about 73%, about 55% to about 70%, about 58% to about 68%, about 60% to about 65%, about 62% to about 64%, or about 63% of sodium polystyrene sulfonate.

The weight of the copolymer and polymers in the mixture is typically greater than 100,000 Daltons and preferably greater than 400,000 Daltons, such that the copolymer is large enough not to be absorbed by the gastrointestinal tract. The amount of oligomers is advantageously minimized, such that there is less than about 0.3%, preferably less than about 0.1%, or more preferably less than about 0.05% (w/w) oligomers. The upper limit of the weight is generally not crucial. Typically, copolymers and polymers of the present invention weigh from about 100,000 Daltons to about 5,000,000 Daltons, or about 200,000 Daltons to about 2,000,000 Daltons, about 300,000 Daltons to about 1,500,000 Daltons or about 400,000 Daltons to about 1,000,000 Daltons. The polystyrene sulfonate copolymer or polymer can either be crosslinked or uncrosslinked, but is preferably uncrosslinked and water soluble.

Another embodiment of the present invention is a polystyrene sulfonate polymer in which at least 10%, 20%, 30%, 35%, 50% or 75% of its countercations are potassium cations. Preferably the polystyrene has at least two different countercations, more preferably only two different countercations, and even more preferably these two countercations are potassium and sodium. Typically, about 20% to about 70% of the counterions are potassium and about 30% to about 80% of the counterions are sodium. Alternatively, about 30% to about 45% of the counterions are potassium and about 55% to about 70% sodium; about 35% to about 40% of the counterions are potassium and about 60% to about 65% of the counterions are sodium; about 37% of the counterions are potassium and about 63% of the counterions are sodium; about 50% to about 60% of the counterions are potassium and about 40% to about 50% are sodium; about 60% to about 70% of the counterions are potassium and about 30% to about 40% are sodium; about 70% to about 80% of the counterions are potassium and about 20% to about 30% are sodium; and about 80% to about 90% of the counterions are potassium and about 10% to about 20% are sodium.

Also included in the present invention are pharmaceutical compositions comprising a pharmaceutically acceptable carrier or diluent and the polystyrene sulfonate polymer described in the prior paragraph. Also included is a method of treating a mammal with AAD or C. difficile associated diarrhea. The method comprises administering to the mammal an effective amount of the polystyrene sulfonate polymer described in the previous paragraph.

Antibiotic associated diarrheas which can be treated by the method of the present invention include, but are not limited to, AADs caused by toxins, such as exotoxins and/or endotoxins produced by Streptococcus spp., including Streptococcus pneumoniae, Streptococcus pyogenes and Streptococcus Sanguis; Salmonella spp., including Salmonella enteritidis; Campylobacter spp., including Campylobacter jejuni; Escherichia spp., including E. coli; Clostridia spp., including Clostridium difficile and Clostridium botulinum; Staphylococcus spp., including Staphylococcus aureus; Shigella spp., including Shigella dysenteriae; Pseudomonas spp., including Pseudomonas aeruginosa; Bordatella spp., including Bordatella pertussis; Listeria spp., including Listeria monocytogenes; Vibrio cholerae; Yersinia spp., including Yersinia enterocolitica; Legionella spp., including Legionella pneumophilia; Bacillus spp., including Bacillus anthracis; Helicobacter spp., including H. pyroli; Corynebacteria spp.; Actinobacillus spp.; Aeromonas spp.; Bacteroides spp. including Bacteroides fragilis; Neisseria spp, including N. meningitidis; Moraxella spp., such as Moraxella catarrhalis and Pasteurella spp. Generally, the AAD is caused by Campylobacter spp., E. coli., S. aureus, P. aeruginosa, V. cholerae, B. fragilis, Neisseria spp., C. novi, C. perfringes, or C. sordelli. Also, AAD may be caused by protozoal toxins, such as toxins produced by Entamoeba histolytica and Acanthamoeba; and parasitic toxins. Typically, the AAD is Clostridium difficile associated diarrhea.

A pharmaceutical composition and methods of treatment of the present invention can optionally include an antibiotic effective against AAD, in addition to the polystyrene sulfonate copolymer or mixture. The antibiotic can be administered simultaneously, for example, in separate dosage forms or in a single dosage form, or in sequence separated by appropriate time intervals. Antibiotics effective against AAD are typically those which are antibacterial, such as those listed in Goodman and Gilman's “The Pharmaceutical Basis of Therapeutics, Ninth Edition,” which is incorporated herein by reference. However, although antibacterial antibiotics will generally treat AAD, effectiveness of many antibiotics against AAD is limited, thereby decreasing the number of possible treatments for a patient suffering from AAD. Preferably, the antibiotic is metronidazole or vancomycin.

The copolymer or polymer can be administered orally or rectally, such as through a feeding tube. Preferably, the copolymer or polymer or the pharmaceutical composition comprising the copolymer polymer is administered orally. The form in which the copolymer or polymer is administered, for example, powder, tablet, capsule, solution, slurry, suspension, dispersion, or emulsion, will depend on the route by which it is administered. Suitable pharmaceutical carriers may contain inert ingredients which do not interact with the compound. The carriers should be biocompatible, i.e., non-toxic, non-inflammatory administration site. Examples of pharmaceutically acceptable carriers include, for example, saline, commercially available inert gels, or liquids supplemented with albumin, methyl cellulose or a collagen matrix. Standard pharmaceutical formulation techniques can be employed, such as those described in Remington's Pharmaceutical Sciences, Mack Publishing Company, Easton, Pa. Methods for encapsulating compositions (such as in a coating of hard gelatin or cyclodextran) are known in the art (Baker, et al., “Controlled Release of Biological Active Agents”, John Wiley and Sons, 1986).

For oral administration, the copolymers and polymers can be formulated readily by combining the copolymers or polymers with pharmaceutically acceptable carriers well known in the art. Such carriers enable the copolymers and polymers of the invention to be formulated as tablets, pills, dragees, capsules, liquids, gels, syrups, slurries, suspensions and the like, for oral ingestion by a patient to be treated. Pharmaceutical preparations for oral use can be obtained by combining the copolymer or polymer with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound or polymer doses.

Pharmaceutical preparations that can be used orally include push-fit capsules made of a suitable material, such as gelatin, as well as soft, sealed capsules made of a suitable material, for example, gelatin, and a plasticizer, such as glycerol or sorbitol. The push-fit capsules can contain the copolymer or polymer in admixture with filler such as lactose, binders such as starches, and/or lubricants such as talc or magnesium stearate and, optionally, stabilizers. In soft capsules, the copolymer or polymer can be dissolved or suspended in suitable liquids, such as aqueous (saline) solutions, alcohol, fatty oils, liquid paraffin, or liquid polyethylene glycols. In addition, stabilizers can be added. All formulations for oral administration should be in dosages suitable for such administration.

An “effective amount” of the present copolymers or mixtures is an amount sufficient to treat (e.g., inhibit), partially or totally, AAD, for example, by ameliorating, delaying the onset, or shortening the duration of the symptoms of AAD, or by inhibiting the relapse of AAD. The effective amount can be administered in a single dose or in a series of doses separated by appropriate time intervals, such as hours.

The quantity of a given polymer or copolymer to be administered will be determined on an individual basis and will be determined, at least in part, by consideration of the size of the individual susceptible mammal, general health, age, sex, body weight, tolerance to pharmaceutical agents, the identity of the known or suspected pathogenic organism, the severity of symptoms to be treated and the result sought. The polymer or copolymer can be administered alone or in a pharmaceutical composition comprising the polymer or copolymer and one or more pharmaceutically acceptable carriers, diluents or excipients. The pharmaceutical composition can also, optionally, include one or more additional drugs, such as antibiotics, anti-inflammatory agents or analgesics.

For oral delivery, copolymers or mixtures (i.e., with respect to the amount of polymer in the mixture) can be administered at a dosage of about 0.1 to about 10 g/day and more preferably from about 1.0 to about 7.0 g/day and even more preferably from about 2.0 to about 6.6 g/day. Most preferably, copolymers or mixtures are administered at a dosage of about 3.0 to about 6.0 g/day.

The polystyrene sulfonate copolymer or polymer mixture, particularly in a pharmaceutical composition, advantageously has less than about 0.1% (w/w) of any of one impurity as measured by gas chromatography, such that the total amount of impurities is less than 0.5% (w/w). In particular, the amount of 1,2-dichloroethane should be less than about 0.0005% (w/w). Also, the amount of residual styrene measured by HPLC should be less than about 0.001% (w/w). The amount of residual chloride and bromide should each be less than about 1.0%, as measured by ion chromatography. Heavy metals preferably constitute less than 0.002% (w/w) of the polystyrene copolymer or polymer mixture. The level of microbes is advantageously minimized, such that there are no more than about 500 colony-forming units (cfu) per gram of aerobic organisms, no more than 250 cfu/g of molds and yeast and there are no detectable pathogens.

Polystyrene sulfonate polymers and copolymers of the present invention can be prepared by the methods previously described. For example, U.S. Pat. Nos. 6,270,755, 6,290,946, 6,419,914, 6,517,826 and 6,517,827 describe methods of synthesis polystyrene sulfonate polymers by polymerizing styrene sulfonate (e.g., Examples 8 and 12 of U.S. Pat. No. 6,290,946). When polymerizing potassium styrene sulfonate and sodium styrene sulfonate, a suitable amount (e.g., about 1 to about 5 equivalents, preferably about 1.8 to about 2.0 equivalents) of sodium styrene sulfonate is polymerized with a suitable amount (e.g., about 1 to about 4 equivalents, preferably about 0.9 to about 1.1 equivalents) of potassium styrene sulfonate, to form a copolymer, preferably a random copolymer, where about 30% to about 80%, about 55% to about 70%, about 60% to about 65%, or about 63% of the repeat units comprise sodium styrene sulfonate and about 20% to about 70%, about 30% to about 45%, about 35% to about 40%, or about 37% of the repeat units comprise potassium styrene sulfonate.

In one method of polymerizing sodium styrene sulfonate and potassium styrene sulfonate involves mixing suitable amounts of the monomers in water (e.g., purified water) and heating the mixture to about 50° C. to about 100° C., preferably about 60° C. to about 90° C., or more preferably about 80° to about 85° C. A catalytic amount of a polymerization initiator (e.g., sodium persulfate, AIBN) is added and the mixture is stirred for at least 4 hours (preferably at least 8 hours) at 50° C. to about 120° C., preferably about 60° C. to about 100° C., or more preferably about 80° to about 90° C. The mixture is then cooled to about 20° C. to about 40° C. One or all of these steps can be conducted under nitrogen or a nitrogen purge.

When exchanging a proportion of the potassium ions of potassium polystyrene sulfonate for sodium ions, typically about 30% to about 80%, about 55% to about 70%, about 60% to about 65%, or about 63% of the potassium ions are exchanged for sodium ions. Alternatively, when exchanging a proportion of the sodium ions of sodium polystyrene sulfonate for potassium ions, typically between about 20% to about 70%, about 30% to about 45%, about 35% to about 40%, or about 37% of the sodium ions are exchanged for potassium ions.

In one example, a proportion of the potassium ions of polystyrene potassium sulfonate can be exchanged for sodium ions by dissolving the potassium polystyrene sulfonate in a solution containing sodium salts or potassium and sodium salts. Examples of sodium salts include sodium chloride, sodium bromide, sodium sulfate, and sodium citrate. In another example, a proportion of the sodium ions of polystyrene sodium sulfonate can be exchanged for potassium ions by dissolving the sodium polystyrene sulfonate in a solution containing potassium ions or potassium and sodium ions. Examples of potassium salts include potassium chloride, potassium bromide, potassium sulfate, and potassium citrate. The solution contains a sufficient quantity of sodium (or potassium) salts in a suitable ratio to achieve the desired sodium/potassium ratio on the polystyrene sulfonate. The above method is also useful for converting the sodium salt of styrene sulfonate to the potassium salt of styrene sulfonate and for converting the potassium salt of styrene sulfonate to the sodium salt of styrene sulfonate.

A proportion of the sodium ions of polystyrene sodium sulfonate can also be exchanged for potassium ions by contacting polystyrene sodium sulfonate with a cationic exchange resin loaded with potassium ions. Similarly, a proportion of the potassium ions of polystyrene potassium sulfonate can be exchanged for sodium ions by contacting polystyrene potassium sulfonate with a cationic exchange resin loaded with sodium ions. The cationic exchange resin contains a sufficient quantity of sodium (or potassium) salts in a suitable ratio to achieve the desired sodium/potassium ratio on the polystyrene sulfonate. The above method is also useful for converting the sodium salt of styrene sulfonate to the potassium salt of styrene sulfonate and for converting the potassium salt of styrene sulfonate to the sodium salt of styrene sulfonate.

Ion exchange processes involving a cationic exchange resin can be carried out in a throw-away mode, a regenerative mode, or in a continuous counter-current mode in simulated moving bed (SMB) equipment. In the throw-away mode, fresh cationic exchange resin is used for each synthesis. In the regenerative mode, after ion exchange is carried out, the cationic exchange resin is contacted with a solution containing sodium and/or potassium ions, such that the ion content of the resin is partially or completely restored to the ion content prior to ion exchange. Such cationic exchange resins can be used in more than one synthetic process. In the continuous counter-current mode, ion exchange is carried out in simulated moving bed equipment, such that regenerant chemical consumption and waste stream are minimized and cationic exchange resins are regenerated as the process continues. The above method is also useful for converting the sodium salt of styrene sulfonate to the potassium salt of styrene sulfonate and for converting the potassium salt of styrene sulfonate to the sodium salt of styrene sulfonate.

A proportion of the sodium ions of polystyrene sodium sulfonate can be exchanged for potassium ions by electrodialysis. Similarly, a proportion of the potassium ions of polystyrene potassium sulfonate can be exchanged for sodium ions by electrodialysis. In electrodialysis, for example, a polystyrene sodium sulfonate solution and a solution containing a potassium salt (e.g., potassium sulfate, potassium chloride) are passed through alternate channels of a stack of cation and/or anion exchange membranes. Conditions such as voltage, current density, flow rate of the solutions, and operation in co- or counter-current mode are controlled to produce a copolymer with the desired sodium and potassium ion content. Electrodialysis can be carried out using commercially available electrodialysis membranes available from, for example, Tokoyama Soda and Asahi. The above method is also useful for converting the sodium salt of styrene sulfonate to the potassium salt of styrene sulfonate and for converting the potassium salt of styrene sulfonate to the sodium salt of styrene sulfonate.

Polystyrene can be sulfonated, for example, by reacting polystyrene with concentrated sulfuric acid, oleum, sulfur trioxide, or a sulfur trioxide/pyridinium complex and warming the mixture (e.g., to 40-50° C. for sulfuric acid, 20-25° C. for oleum). The resulting polystyrene sulfonic acid can be washed extensively, for example, with water, until the pH increases to 4 to 5. The polystyrene sulfonic acid is preferably neutralized (partially or, more preferably, completely) with an appropriate basic sodium salt, basic potassium salt, or a mixture thereof. When reacting polystyrene sulfonic acid with a mixture of basic sodium and potassium salts, typically about 30% to about 80%, about 55% to about 70%, about 60% to about 65%, or about 63% of the mixture is one or more basic sodium salts and about 20% to about 70%, about 30% to about 45%, about 35% to about 40%, or about 37% of the mixture is one or more basic potassium salts. Basic sodium salts include, for example, sodium hydroxide, sodium carbonate, and sodium bicarbonate. Basic potassium salts include, for example, potassium hydroxide, potassium carbonate, and potassium bicarbonate.

Copolymers synthesized by any of the previously described methods can be purified by ultrafiltering the copolymer. Typically, ultrafiltration occurs simultaneously with or following ion exchange. For processes involving electrodialysis, ultrafiltration typically occurs prior to electrodialysis. Ultrafiltering a copolymer typically includes one or more cycles of diluting and concentrating the copolymer, whereby ions not bound to the copolymer, oligomers, and other contaminants are forced through a membrane (e.g., a membrane that allows passage of molecules and ions having a molecular weight from less than 10,000 kDa to 300,000 kDa) and removed during concentration. Ultrafiltration can be carried out with apparatus that are commercially available from, for example, Millipore, Sartorius, and Pall. The above method is also useful for converting the sodium salt of styrene sulfonate to the potassium salt of styrene sulfonate and for converting the potassium salt of styrene sulfonate to the sodium salt of styrene sulfonate, provided that appropriately-sized membranes are used.

In one ultrafiltration method, a solution of a sodium/potassium polystyrene sulfonate copolymer is optionally diluted with water (e.g., purified water) to give a solution containing about 1% to about 3% (e.g., about 1.5% to about 2.5% or about 2%) by weight of the copolymer. The diluted solution is heated to about 40° to about 50° C. During the ultrafiltration, the retentate is recycled to purify the copolymer over multiple cycles. Water is added in order to maintain an approximately constant volume. The pH is also monitored, such that a pH of approximately 10 (or greater) is maintained. A base (e.g., sodium or potassium hydroxide) can be added if the pH falls below 10. Once the desired purity is obtained (measured by the conductivity of the solution, preferably the conductivity is less than about 250 microS/cm), the solution is concentrated to obtain a solution containing about 3% to about 6% by weight (e.g., about 4%) of copolymer. The pH should still be monitored and adjusted, if necessary, during the concentration. The solution can optionally be further concentrated by vacuum distillation, in order to obtain a solution containing about 8% to about 15% (e.g., about 10%) by weight of copolymer. In one example, the temperature does not exceed about 50° C. during vacuum distillation. In another example, the temperature does not exceed about 80° C. during vacuum distillation.

The concentrated or distilled solutions of copolymers can be dried to obtain the solid copolymer using conventional techniques known to one or ordinary skill in the art. Typically, drying continues until any further weight loss on drying is less than about 10%. The dried copolymer can then be formulated into a pharmaceutical composition. Alternatively, the copolymer solutions can be formulated into a pharmaceutical composition.

### EXEMPLIFICATION

### Example 1

Protection of Vero Cells From Cytotoxicity Caused by C. difficile Toxins A and B

Confluent monolayers of Vero cells (ATCC#CCL-81) were prepared in 96 well microtitre trays. Purified C. difficile toxins A or B were obtained from TechLab (TechLab, Blacksburg Va.). The monolayers were incubated with C. difficile toxin A (10 ng/ml) or toxin B (1 ng/ml) in the presence of serial dilutions of polymers. These toxin concentrations were previously found to cause 100% cell rounding in 18-24 hours. Cells were observed at 24 hours and scored for cell rounding. The concentration of polymer that provided 100% protection from cell rounding is reported in Table 1. Results represent means of duplicate wells.

TABLE 1
Polymer concentration providing 100% protection of Vero Cell
monolayers from toxin A and toxin B mediated cell rounding.

Concentration of polymer
(mg/ml) providing 100%
protection from
toxin A or toxin B

| Polymer | Toxin A | Toxin B |
|---|---|---|
| Sodium Polystyrene | 0.0038-0.0078 | 1.25 |
| Sulfonate | | |
| Poly (Potassium and | 0.0039-0.0078 | 1.25 |
| Sodium Styrene Sulfonate) | | |

Confluent monolayers of Vero cells (ATCC#CCL-8 1) were prepared in 96 well microtitre trays. Purified C. difficile toxins A or B were obtained from TechLab (TechLab, Blacksburg Va.). Monolayers were incubated with serial dilutions of C. difficile toxins A or B in the presence of 10 mg/ml of polymer. The cells were observed for cell rounding at 24 hours. The highest concentration of toxins A and B that was completely neutralized by polymer (no rounding of monolayer) is reported in Table 2. Results represent means of duplicate wells.

TABLE 2
Maximal Toxin Concentration Neutralized by Polymers

| | Maximum concentration neutralized | |
|---|---|---|
| | by 5 mg/ml polymer | |
| | Sodium Polystyrene | Poly (Potassium and Sodium |
| Treatment | Sulfonate | Styrene Sulfonate) |
| Toxin A (ng/ml) | 10 ng/ml | 10 ng/ml |
| Toxin B (ng/ml) | 0.031 ng/ml | 0.031 ng/ml |

### Example 2

Preparation of Sodium/Potassium Polystyrene Sulfonate by Potassium Chloride Addition and Ultrafiltration

Dry solid sodium polystyrene sulfonate powder was dissolved in deionized water to produce 500 g of a 1% w/w polystyrene sulfonate solution. Potassium chloride (1.032 g) was added to the solution, which was then subjected to ultrafiltration (UF). UF involved concentrating the solution from 1% w/w to 2% w/w polystyrene sulfonate five times using a 300 kDa cut-off membrane, and diluting the solution to 1% w/w polystyrene sulfonate between steps with deionized water. The UF process was run at a temperature between 40 EC and 60 EC.

The product of this synthesis was analyzed by inductively-coupled plasma optical emission spectrometry (ICP-OES). Samples were analyzed using direct infusion ICP-OES analysis against a NaCl/KCl calibration curve, as 1:50 diluted neat samples and after ultracentrifugation (30 minutes at 14000× g through a 10_kDa Nanosep filter). ICP-OES analysis showed that 37% of the exchangeable ions were potassium ions.

### Example 3

Preparation of Sodium/Potassium Polystyrene Sulfonate Copolymer

A reactor was filled with 200 L purified water, followed by 26.2 kg sodium styrene sulfonate and 15.1 kg potassium styrene sulfonate. The contents of the reactor were heated to about 800 to 85° C. to form a solution. A solution of 57 g sodium persulfate in 1 L purified water was added to the reactor to form the sodium/potassium polystyrene sulfonate copolymer. The contents of the reactor were stirred for about 21 hours at a temperature of about 80° to 90° C. The contents of the reactor were then cooled to about 32° C.

The contents of the reactor were emptied into a drum and approximately one-eighth of the solution (30 kg) was added back into the reactor, and diluted with 200 L purified water. This mixture was stirred for about 30 minutes and was then emptied into a drum. This dilution step was repeated for the other seven approximately 30 kg portions of the solution.

Approximately half of the diluted solution (932 kg) was added to a reactor, which was purged with a nitrogen bleed of about 5 L/min. With stirring, the diluted solution was heated to between 40° and 50° C. The polystyrene sulfonate copolymer was purified by ultrafiltration. The volume of the diluted solution was kept approximately constant by the addition of 1554 L of purified water during ultrafiltration. The pH of the solution was monitored throughout the entire ultrafiltration procedure to maintain about pH 10. After purification was completed, the purified polystyrene sulfonate (PSS) copolymer solution was concentrated using the ultrafiltration membrane to give an approximately 4% w/w solution of the PSS copolymer, continuing to monitor the pH (40 mL of a 32% w/w NaOH solution was added at the end of concentration). The final volume of the purified PSS copolymer solution was about 400 L, which was cooled to below 40° C. The same purification step was conducted for the remaining half of the diluted solution, although no NaOH was added.

The two concentrated PSS copolymer solutions were combined in the reactor. The solutions were further concentrated by vacuum distillation at about 80° C., to reduce the volume by about 425 L (obtaining about 428 L further concentrated solution). The further concentrated solution contained about 10% w/w of the PSS copolymer. The pH was checked and determined to be about pH 10.3. The further concentrated solution was cooled to below 40° C.

### Example 4

Manufacture and Purification of Sodium/Potassium Polystyrene Sulfonate

Approximately 950 L of purified water are added to a vessel, along with about 100 kg of an approximately 20% (w/w) aqueous solution of sodium polystyrene sulfonate (Na PSS). The mixture is agitated at room temperature until the Na PSS solution is dissolved. A sample is taken to analyze the content of Na PSS.

Potassium chloride (approximately 4.4 kg) is added to the mixture, which is agitated vigorously for about 10 minutes to prepare an approximately 2% (w/w) solution of sodium/potassium polystyrene sulfonate (Na/K PSS). The pH of the Na/K PSS solution is measured, and is adjusted to between pH 10 and 11 (preferably 10.75) with a basic solution of 1 L purified water, 200 g 85% KOH and 330 g NaOH pellets. A sample is taken again to measure the Na/K PSS content and the ratio of sodium to potassium in the solution. The solution is passed through a 0.5 micrometer filter.

These steps are repeated twice to prepare approximately 2000 L of a 2% Na/K PSS solution.

The 2% Na/K PSS solution is heated to between 40° and 50° C. and ultrafiltration is begun. (The ultrafiltration unit is treated with alkali and washed before the purification begins.) At the beginning and end of the ultrafiltration process, the pH of the solution is measured. The pH is adjusted with the basic solution prepared above to between pH 10 and 11 (target 10.75). When the amount of permeate reaches approximately 1050 L, a sample is taken from the Na/K PSS solution to analyze the Na/K PSS content. A cycle of the ultrafiltration process is complete when the Na/K PSS content becomes 4.0±0.2%. After the fourth cycle of the ultrafiltration process, the sodium/potassium ratio and the salt content in the solution is measured. The ultrafiltration process is repeated until the salt content in the permeate is reduced to the desired level. If the salt content remains too high, then approximately 1050 L purified water is added to the retentate before the next ultrafiltration cycle.

When the desired salt content is obtained, the approximately 4% (w/w) Na/K PSS solution resulting from the final ultrafiltration cycle is concentrated to a 9±1% (w/w) solution. The pH is measured again following concentration, and adjusted to between pH 10 and 11 (target 10.75) with the basic solution prepared above. The solution is heated to approximately 80° C. and the temperature is maintained for over 1 hour. The solution is cooled.

### Example 5

Preparing of Sodium/Potassium Polystyrene Sulfonate by Electrodialysis

The electrodialysis process was carried out using 2 L of 2% (by weight) solution of sodium polystyrene sulfonate (NaPSS) as the feed solution. The concentrate solution consisted of 2 L of a 5 g/L NaCl aqueous solution. An aqueous 0.1 eq/L KCl solution was used as the diluate.

The electrodialysis membrane stack was made of five cells, each of which contained alternating cation, anion, and cation membranes. The total effective cell area was 0.1 m².

Electrodialysis was run in batch mode at a constant current density of 10 mA/cm². The temperature of the NaPSS solution was kept at 55° C. The three solutions (feed/product, diluate, and concentrate) were circulated through the appropriate cell channels at approximately 120 L/hr. During electrodialysis, the conductivities of the three streams were monitored.

After the current was passed through the electrodialysis membrane stack for 14 minutes, the process was deemed complete. Analysis of a sample of the product solution showed 35 mol % potassium ions. Two similar repeat experiments showed the potassium content to be 36% and 38%.

### Example 6

Preparing of Sodium/Potassium Polystyrene Sulfonate Using a Cation Exchange Resin

An ion exchange resin bed was prepared by placing 200 ml of strong acid cation resin in sodium form in a 3 cm diameter glass column. The resin was converted into the potassium form by slowly passing 1 L 1.6 N KCl solution through it. The resin was thoroughly washed with deionized water until the effluent showed a negligible amount of chloride.

The ionic conversion process was carried out by slowly (approximate flowrate: 5 ml/min) passing 4.25 L of a 4% (by weight) solution of sodium polystyrene sulfonate (NaPSS) through the resin bed. An additional 1 L of deionized water was used to wash the bed. The total collected effluent showed that the PSS contained 40 mol % potassium (and 60 mol % sodium) following ion exchange. The recovery of the sodium/potassium polystyrene sulfonate copolymer product was greater than 95%.

The resin was further washed with 1 L of deionized water and regenerated with 720 ml of 1.6 N KCl solution. The resin was thoroughly washed with deionized water until the effluent showed a negligible amount of chloride.

Another aliquot of 4.25 L of the 4% (by weight) NaPSS solution was slowly passed through the regenerated resin bed. An additional 1 L of deionized water was used to wash the bed. The total collected effluent showed that the PSS contained 41 mol % potassium. The recovery of the product PSS-Na/K was greater than 95%.

These results demonstrate that the ion exchange resin can be used in a cyclic process consisting of partially converting the NaPSS to the sodium/potassium polystyrene sulfonate copolymer and regenerating the resin.

While this invention has been particularly shown and described with references to preferred embodiments thereof, it will be understood by those skilled in the art that various changes in form and details may be made therein without departing from the scope of the invention encompassed by the appended claims.

1. A polystyrene sulfonate copolymer, wherein the copolymer is comprised of repeat units represented by Structural Formula (I): 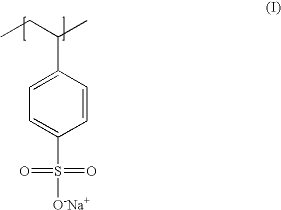
and repeat units represented by Structural Formula (II):

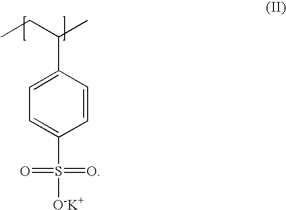

2. The copolymer of claim 1, wherein the copolymer consists of repeat units represented by Structural Formula (I) and Structural Formula (II).

3. The copolymer of claim 1, wherein 30% to 80% of the repeat units are represented by Structural Formula (I) and 20% to 70% of the repeat units are represented by Structural Formula (II).

4. The copolymer of claim 3, wherein the copolymer has a molecular weight ranging from 100,000 to 5,000,000 Daltons.

5. The copolymer of claim 4, wherein the copolymer has a molecular weight of 300,000 to 1,500,000 Daltons.

6. The copolymer of claim 3, wherein the repeat units are uncrosslinked.

7. A pharmaceutical composition comprising a pharmaceutically acceptable carrier and a polystyrene sulfonate copolymer, wherein the copolymer is comprised of repeat units represented by Structural Formula (I): 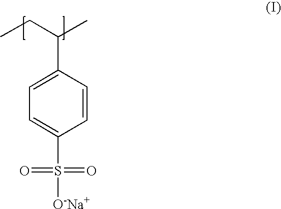
and repeat units represented by Structural Formula (II):

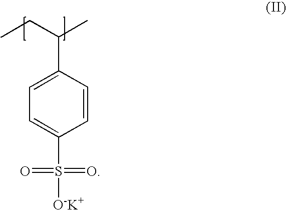

8. The pharmaceutical composition of claim 7, wherein 30% to 80% of the repeat units are represented by Structural Formula (I) and 20% to 70% of the repeat units are represented by Structural Formula (II).

9. The pharmaceutical composition of claim 8, further comprising an antibiotic effective against antibiotic associated diarrhea.

10. The pharmaceutical composition of claim 9, wherein the antibiotic is metronidazole or vancomycin.

11. A polystyrene sulfonate polymer in which at least 10% of its counteractions are potassium cations.

12. The polymer of claim 11 wherein the polymer has two different counteractions.

13. The polymer of claim 12 wherein the two counteractions are potassium and sodium.

14. The polymer of claim 11 wherein between about 20% to about 70% of the counteractions are potassium and about 30% to about 80% are sodium.

15. The polymer of claim 11 wherein between about 35% to 40% of the counteractions are potassium and about 60% to 65% are sodium.
